(19) United States
(12) Patent Application Publication
(10) Pub. No.: US 2003/0228272 A1
(43) Pub. Date: Dec. 11, 2003

(54) Novel antidandruff conditioning shampoo

(76) Inventors: Zahid Amjad, Downers Grove, IL (US); Archish Desai, Morton Grove, IL (US)

Correspondence Address: George W. Moxon II, Esq., Roetzel & Andress, 222 South Main Street, Akron, OH, 44308, US

(21) Appl. No.: 10/397,155

(22) Filed: Mar. 26, 2003

Related U.S. Application Data
(60) Provisional application No. 60/368,437, filed on Mar. 27, 2002.

(51) Int. Cl. A61K 7/075; A61K 7/08; A61K 31/555

(52) U.S. Cl. 424/70.28; 514/184

(57) ABSTRACT

A novel antidandruff conditioning shampoo comprising a cleansing surfactant, a water-insoluble antidandruff and/or anti-itching agent, a conditioning agent, a natural oil, and a suitable carrier, and a method of treating hair are disclosed. The shampoo compositions effectively suspend the water-insoluble hair treating compound, effectively cleanse the hair and effectively deliver the water soluble and water insoluble hair-treating compounds to the hair and scalp and also effectively condition the hair.

### CROSS REFERENCE TO RELATED CLAIMS

This application claims the benefit of the filing of U.S. Provisional Patent Application Serial No. 60/368,437, entitled “Novel Antidandruff Conditioning Shampoo”, filed on Mar. 27, 2002, and the specification thereof is incorporated herein by reference.

### BACKGROUND OF THE INVENTION

The present invention relates generally to anti-dandruff shampoos which include a conditioner and, more particularly, to the inclusion of natural oils, such as tea tree oil in shampoos which also include an antidandruff agent such as zinc pyrithione.

Human hair becomes soiled and dirty due to its contact with the surrounding environment and, to a greater extent, from sebum secreted by the head and the impurities present in the air. The build-up of unwanted deposits causes the hair to have a dirty and an unattractive appearance. The soiling of the hair therefore necessitates the use of shampoo with frequent regularity to keep the hair clean.

Shampooing the hair cleans by removing excess soil and sebum. Hair shampoos generally are formulated with highly effective synthetic surfactants, like anionic surfactants, that primarily cleanse as opposed to conditioning and/or treating the hair. Shampoos usually neither aid in the detangling of wet hair nor impart any residual conditioning benefits to dry hair, such as manageability or style ability of hair sets. Thoroughly cleansed hair when done with anionic surfactant-based shampoo, leaves the hair with an undesirable harsh, dull and dry touch, or feel, and are extremely difficult to comb and/or brush in either wet or dry state. Incompletely dried hair, such as by using a towel, has poor combing and/or brushing properties and, after complete drying, does not set well. This unsatisfactory combing or brushing property immediately after shampooing also causes hair damage, such as split ends or hair breakage. In addition, the natural luster and resiliency of the hair is reduced.

The overall unsatisfactory condition of shampooed hair usually necessitates a subsequent post-shampoo treatment of the hair with a conditioning composition to improve these undesirable physical characteristics. These conditioning compositions normally are applied separately from the hair shampoo, and usually are rinses or cream-like lotions containing a cationic compound that is substantive to the hair. However, there are disadvantages associated with such conditioning products. With respect to hair rinses, they must remain on the hair for a length of time and then the hair must be rinsed with fresh water, an additional time. With respect to leave-on hair conditioners, the amount of conditioner to apply is often difficult to determine based on the amount of thickness of the hair to be conditioned, and even then the uniform distribution of conditioner throughout the hair can be a problem.

As a result, consumers often desire a hair shampoo that can take care of all their needs by imparting other desirable properties, in addition to cleaning, to the hair or scalp, such as conditioning, as well as antidandruff and set retention properties. Therefore, to cope with the above problems, it became desirable to formulate a combined shampoo and conditioner.

Although shampoos and conditioners in theory could be combined in the same product, in actuality problems arose concerning the compatibility between the detergents and conditioners present in the product. This problem was addressed by the development of suspension agents. For example, U.S. Pat. No. 4,741,855 to Grote et al discloses a shampoo composition comprising a detergent, a silicone conditioner, water and a suspension agent, such as long chain esters of ethylene glycol and esters of long chain fatty amine oxides. Grote et al do not teach a conditioning shampoo which is also anti-dandruff shampoo.

Other examples of patents teaching conditioning shampoos include U.S. Pat. No. 5,015,415 to Groze et al that discloses a conditioning shampoo comprising a certain phthalic acid and phthalic acid ammonium salts, or mixtures thereof, incorporated into a shampoo base with a silicone conditioner. In Cosmetic Toiletry, and Fragrance Association (CTFA) nomenclature, phthalic acids may be designated as amido carboxy benzoic acids, and phthalic acid ammonium salts may be designated ammonium amido carboxy benzoates. U.S. Pat. No. 4,472,375 to Bolich et al. Teach an aqueous hair conditioning composition comprising a volatile hydrocarbon or a volatile silicone; a nonionic thickening agent; a quaternary ammonium salt and/or salt of a fatty amine. The composition of Bolich et al. does not include an anionic cleansing surfactant and relies upon the nonionic thickening agent, e.g., a polymer, to suspend the water-insoluble ingredients.

Consumers also often use an antidandruff shampoo. The incorporation of antidandruff agents into anionic surfactant-based shampoo is well known. The antidandruff agents not only must relieve the flaking and itching symptoms of dandruff, but also must be substantive to the skin and hair in order to extend the efficacy of the antidandruff agent from one shampoo treatment to the next. These properties are most often found in compounds that are insoluble in aqueous media, and this inherent poor solubility of the antidandruff agent poses a difficult problem in developing a stable aqueous anionic-based surfactant shampoo.

In order to incorporate effective, water-insoluble antidandruff agents into aqueous surfactant-based hair shampoo, one or more suspending agents are required to keep the antidandruff agent homogeneously dispersed throughout the aqueous solution. A water-insoluble particulate antidandruff agent poses a formulation stability problem, as antidandruff agent tends to separate out from the aqueous hair shampoo base. Failure to adequately suspend the antidandruff agent leads to eventual shampoo separation as the antidandruff agent settles to the bottom of the container, and results in poor dandruff control and consumer complaints. As a result, scientists have continuously researched for suitable suspending agents capable of effectively dispersing antidandruff agents such as selenium sulfide, sulfur, zinc pyrithione, coal tar, and salicylic acid in aqueous media. The suspending agent can be any one of a number of inorganic minerals or natural polymers or gums. Among the most often used suspending agents are colloidal aluminum oxide, modified magnesium aluminum silicate, lignins, xanthan gum, fumed silica, algin products, polyacrylic acid, sodium carboxymethyl cellulose, hydroxylpropyl cellulose, and alkanolamides. For example, U.S. Pat. No. 5,587,154 to Dowel et al discloses the use of amine-based compounds as suspending agents for the delivery of antidandruff agents such as selenium sulfide, salicylic acid, and zinc pyrithione. U.S. Pat. No. 4,927,563 to McCall discloses the use of combination of xanthan gum and magnesium aluminum silicate as a suspension system for the improved deposition of selenium sulfide in antidandruff shampoos. U.S. Pat. No. 5,624,666 to Coffindaffer et al. teaches a composition that is efficacious for dandruff treatment while being substantially free from suspending agents.

The use of natural oils in personal care products is known. Typically these uses include cosmetics and washes, but they also include hair care, such as rinses and/or conditioners that are uses after the hair is washed. For example, U.S. Pat. No. 6,039,937 to Neubauer discloses a liquid composition comprising vegetable oil, vitamin E and tea tree oil for treating razor and razor blades between uses to enhance the useful life of razor or razor blade. U.S. Pat. No. 6,123,982 to Fontana discloses the use of tree tea oil as flavoring agent in dental floss. U.S. Pat. No. 6,140,284 to Cheung et at. teaches the use of botanical oils, which includes a long list of “essential oils”, including tea tree oil, as blooming agents in hard surface cleaning compositions.

### SUMMARY OF THE INVENTION

The present invention is the result of the discovery that the addition of a natural oil to an antidandruff shampoo that includes a conditioner produces an improved shampoo which provides the user with reduced dandruff flaking and less itching and which exhibits excellent cosmetic attributes such as lathering and conditioning, as compared to previous antidandruff shampoos which contain conditioners. In addition, the antidandruff shampoo provides decreased frequency of scalp itching between shampooing, decreased dandruff flaking, easy removal of dandruff flakes from hair, and the composition is acceptably stable.

The shampoo composition comprises, essentially, in percent by weight, from about 10% to about 45% of a synthetic surfactant, from about 0.001% to 5% of particulate antidandruff agent, 0.001% to 5% conditioning agent, 0.001% to 10% natural oil, and enough of a suitable carrier, such as water, to total 100% by weight. The shampoo can contain optional components, which are described in detail below.

The present composition does not rely on a thickening agent to maintain phase stability of the composition. The antidandruff agent, as well as the additional suspending agents added to the basic hair shampoo base, add antidandruff properties to the shampoo without compromising the detergency performance and aesthetic appeal of the shampoo or adversely affect the foaming characteristics of the shampoo composition.

### DETAILED DESCRIPTION OF THE INVENTION

As noted, the present invention is a conditioning, anti-dandruff shampoo which essentially consists of the following ingredients which are employed in the following amounts, with all percentages being by weight and totaling 100%:

(a) Surfactant(s)—10% to 45%,

(b) Foam boosters and/or stabilizer—0% to 6%, preferably 2% to 6%,

(c) Conditioning agent—0.001% to 5.0%,

(d) Antidandruff agents—0.01% to 5.0%, preferably 0.3% to 2.0%, with about 1.0% being further preferred,

(e) Natural oils—0.001% to 10.0%, preferably 0.3% to 5.0%, with about 1.0% being further preferred,

(f) Preservatives—An optional ingredient, but if used then in an effective amount, i.e., sufficient to retard the degradation of the final composition in order to provide adequate shelf life,

(g) Optional ingredients, as desired—0% to 10.0%, preferably 0.1% to 10.0%,

(h) Acid, base or buffer—if necessary, an effective amount to yield a pH in the desired range,

(i) Non-aqueous solvent—0% to 10%, preferably 0.01% to 10%, and

(j) Water QS ad 100%, that is, sufficient water to make 100%.

Surfactant

An essential component of the present compositions is a surfactant. The surfactant may be selected from any of a wide variety of synthetic anionic, amphoteric, zwitterionic and nonionic surfactants and is present in an amount of from about 5% to about 40%, preferably from about 10% to about 45%, more preferably from about 15% to about 30%.

The synthetic anionic surfactants can be alkali metal salts of organic sulfuric reaction products having in their molecular structure an alkyl radical containing 8-22 carbon atoms and a sulfonic acid or sulfuric acid ester radical (included in the term alkyl is the alkyl portion of higher acyl radicals). Preferred are the sodium, ammonium, potassium or triethylamine alkyl sulfates, especially those obtained by sulfating the higher alcohol (C₈ to C₁₈ carbon atoms), sodium coconut oil fatty acid monoglyceride sulfates and sulfonates; sodium or potassium salts of sulfuric acid esters of the reaction product of 1 mole of a higher fatty alcohol (e.g., tallow or coconut oil or alcohols) and 1 to 2 moles of ethylene oxide; sodium or potassium salts alkylphenol ethylene oxide ether sulfate with 1 to 10 units of ethylene oxide per molecule and in which the alkyl radicals contain from 8 to 12 carbon atoms, sodium alkyl glyceryl ether sulfonates; the reaction product of fatty acids having from 10 to 22 carbon atoms esterified with isethionic acid and neutralized with sodium hydroxide; water soluble salts of condensation products of fatty acids with sarcosine; and the like surfactants.

In conjunction with the anionic surfactant, an amphoteric surfactant can be included in the shampoo composition. An amphoteric surfactant enhances skin mildness and composition esthetics to a improve consumer acceptance. Suitable classes of amphoteric surfactants included in the present invention include, but not limited to, betaines, hydroxpropylsultaines, amine oxides and combinations thereof. Example of specific amphoteric surfactants include, but not limited to, cocamidopropyl betain, lauramidopropyl betain, coco/oleamidopropyl betain, coco betain, oleyl betain, cocamidopropyl hydroxysultaine, tallowamidopropyl hydroxysultaine and dihydroxyethyl tallow glycinate or combinations thereof. In general, however, any amphoteric surfactant known and used in the art of hair shampoos can be included in the composition of the present invention.

The shampoo composition of the present invention can also include a nonionic surfactant in combination with the anionic surfactant. In general, nonionic surfactants impart esthetic, physical or cleansing properties to the shampoo composition. Representative nonionic surfactants that can be included in shampoo composition of the present invention include ethers of polyols and sugars; fatty acid alkalnolamides; polyethylene glycols; the ethoxylated or propoxylated alkylphenols; ethoxylated or propoxylated fatty alcohols; and the condensation products of ethylene oxide with long chain amides. These nonionic surfactants, as well as numerous others not cited herein, are well known to person skilled in the art and are fully described in the literature, such as McCUTHEON's DETERGENT AND EMULSIFIERS, 1989 Annual, published by McCutheson Division, MC Publishing Company, which is incorporated herein by reference.

In particular, a nonionic alkanolamide can be included in the composition to improve composition thickening and foam stability. Suitable alkalnolamides include, but not limited to, those known in the art of hair care formulations, such as cocamide MEA, cocamide DEA, soyamide DEA, lauramide DEA, oleamide MIPA, stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA and combinations in accordance with an important feature of the present invention, the anionic surfactant is present in the composition in an amount of about 5% to about 40%, and preferably about 5% to about 30%, by weight of the composition.

Many cationic surfactants are known to the art. By way of example and not intending to be limited, the following may be used:

Dodecyltrimethyl ammonium chloride

Monylbenzylethyldimethyl ammonium nitrate

Tetradecylpyridinium bromide

Laurylpyridinium chloride

Cetylpyridinium chloride

Laurylisoquinolium bromide

Ditallow(hydrogenated)dimethyl ammonium chloride

Stearylalkonium chloride

Many additional non-soap surfactants are described in McCUTHEON's DETERGENTS AND EMULSIFIERS. The above-mentioned surfactants can be used alone or in combination in the shampoo compositions of the present invention.

Conditioning Agents

These agents are used in shampoos, especially in the conditioning shampoos, to provide conditioning effects to the hair. A large number of materials would be suitable conditioning agents for shampoos. Typically, a blend of conditioning agents is employed, as is typical in the shampoo formulating art. Preferred conditioning agents are the following types of materials:

Silicones

The use of silicon compounds as conditioning agents in shampoos is well known, and they are employed in their intended use. The volatile silicone normally is a low molecular weight polydimethylsiloxane, however a low molecular weight polysiloxane including phenyl substituents also is useful in the present invention. Furthermore, the low molecular weight polydimethysiloxane compound can be a linear or a cyclic polydimethysiloxane compound. The volatile polydimethylsiloxane compound provides lubrication and imparts hair-conditioning properties to wet hair, and has sufficient volatility to slowly volatilize from the hair such that a residual buildup of silicone compound is not present on dry hair.

The silicone compounds that are commonly used in shampoos are dimethicone, dimethiconepolyol, and silicone gum. Dimethicone belongs to the class of compounds known as polydimethylsilooxanes. Dimethicone coplyol is a derivative of dimethicone in which polyether side chains replace some of the methyl groups. Silicone gum is mixed high molecular weight (from 200,000 to 1,000,000) dimethicone compound that is gum like in nature. Dimethicone and dimethicone polyol can be represented by the following formula:

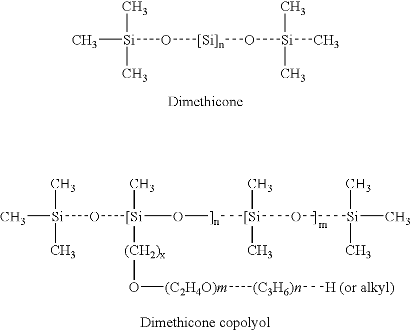

In addition, the cyclic, low molecular weight, volatile polydimethylsiloxanes, having the Cosmetic, Toiletry and Fragnance Association (CTFA) designation cyclomethicones, are also useful in the composition and method of the present invention. The cyclomethicones are low molecular weight, water-insoluble cyclic compounds having an average of about 3 to 6—[O—Si(CH₃)₂]—repeating group units per molecule and boil at atmospheric pressure in a range of from about 150° C. to about 250° C. Suitable cyclomethicones are available commercially under the trade names SILICONE SF-1173 (octamethylcyclotetrasiloxane) from General Electric, Waterford, N.Y., and SILICONE 334 FLUID and SILICONE 345 FLUID fro Dow Corning Corporation, Midland, Mich., the tetramer being listed first in each instance. The volatile cyclic silicones can be used in combination with a linear volatile silicone conditioner, and the volatile silicone conditioner can be used in conjunction with the nonvolatile silicone conditioner.

Another suitable water-insoluble conditioning compound that can be included in the composition of the present invention is a nonvolatile hydrocarbon, such as mineral oil. The nonvolatile hydrocarbons provide many of the same benefits as the silicone conditioning agents, and can be included in the composition with a silicone conditioning agent. Other oils such as almond oil, avacado oil, sheabutter, aloe vera, etc., can also be used.

The uses of cationic polymers, which have quaternized nitrogen attached to the backbone of the polymer, in hair care products are well known. Being cationic, these polymers are also substantive to hair and are able to impart conditioning attributes to the hair. Suitable cationic polymers include, for example, copolymers of vinyl monomers having cationic amine or quaternary ammonium functionalities with water soluble spacer monomers such as acrylamide, methacrylamide, alkyl and dialkylmethacrylamide, alkyl acrylate, alkyl methacrylate, vinyl caprolactone, and vinyl pyrrolidone. The alkyl and dialkyl substituted monomers preferably have C1-C7 alkyl groups, more preferably C1-C3 alkyl groups. Other suitable spacer monomers include vinyl esters, vinyl alcohol (made by the hydrolysis of vinyl acetate,) ethylene glycol, propylene glycol, and maleic anhydride.

The quaternized polymers useful as conditioning agents are poly(quaterniu-7 (commercial name Merquat 550 available from Calgon); polyquaternium-10 (commercial name Polymer JR, available from Amerchol) polyquaternium-11 (commercial 734 and Gafquat 755), which can be represented by the following structures:

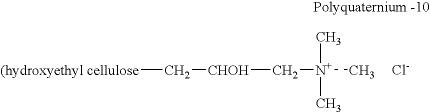

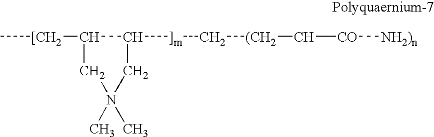

The cationic polymers hereof will generally have a weight average molecular weight which is at least about 20,000. Typically at least about 50,000, and is less than about 10 million. Preferably, molecular weight to be in the range from 50,000 to 1 million. The cationic polymers will have cationic nitrogen-containing moieties such as quaternary ammonium or cationic amino moieties, or mixtures thereof.

Other cationic polymers that can be used include cationic guar gum derivatives, such as guar hydroxypropyltrimonium chloride (commercially available from Celanese Corp. in Jaguar R series). Other materials include quaternary nitrogen containing cellulose ethers (e.g., described in U.S. Pat. No. 3,962,418, incorporated by reference herein) and copolymers of etherified cellulose and starch (e.g., as described in U.S. Pat. No., 3,958,581, incorporated by reference herein.

Antidandruff Agent

The antidandruff agent is normally a water-insoluble hair treatment compound and usually is a particulate compound that is capable of relieving the symptoms of dandruff and is substantive to hair and scalp to impart residual antidandruff properties between shampoos. Examples of particulate compounds exhibiting antidandruff properties include, but not limited to, elemental sulfur, salicylic acid, seleneium sulfide, coal tar preparations, zinc pyrithione, a water-insoluble1-hydroxy pyridone, an azole antimycotic, and undecylenic acid and mixtures thereof. Particularly advantageous antidandruff agents useful in the shampoo composition of the present invention are zinc pyrithione and elemental sulfur. Zinc pyrithione is the zinc complex of 2-pyridinethiol-1-oxide, and is available commercially from Arch Chemicals, under the brand name of Omadine®. Useful sulfur containing compounds include elemental sulfur and selenium sulfide of sufficient purity and particle size to function as an antidandruff agent, as is well known to those skilled in the art. The anti dandruff agent is used in an amount of about 0.01% to about 5.0%, preferably 0.3% to 2.0%, with about 1.0% (±10%) being further preferred.

Natural Oils

While oils and oil derivatives, synthetic as well as natural, can be employed as other ingredients as is commonly done in skin and hair care products to impart conditioning properties to hair, tea tree oil, in the present invention, functions in excess of its conditioning properties and appears to produce a more that additive result when used in combination with anti-dandruff agents. Commonly used oils include canola, olive, castor, avocado, almond, soybean, grape seed, hazelnut, and others. The use of oils in shampoos has found limited applicability due to their incompatibility with the surfactants. The incompatibility results in poor foaming, lather, and cleansing properties.

Tea tree oil is a bioactive essential oil, produced through the steam distillation of the foliage of Melaleuca alternfolia, an Australian native plant of the Mytaceae family, indigenous to the north coast of New South Wales. There are over 220 species of Melaleuca and six chemotypes of Melaleuca alternfolia. The oil is extracted by steam distillation of the foliage and contains a combination of monoterpenes, sesquiterpenes, and terpene alcohols. The principal component is Terpinen-4-ol and is present in about 30% to 45% by weight. The preferred composition is Main Camp Australian Standard Grade which meets Draft International Standard 4730. The term “tea tree oil” is intended to cover equivalent oils whether natural or artificial, which have the same functionality. It has been reported that tea tree oil possesses 48 organic compounds, containing both antiseptic and antifungal properties. Tea tree oil has also been reported to have an anti-inflammatory action. The natural oils are employed in an amount of about 0.001% to about 10.0% by weight, preferably 0.01% to 5.0%, with about 1.0% being further preferred.

Pearlescent Agent

Another desirable component of the shampoos herein is a pearlescent material. Such materials are well known ion the art and include bismuth oxychloride, stearic monoethanolamide, ethylene glycol mono stearate or distreate, guanine and titanium dioxide coated mica. A pearlescent material is generally present at a level of from about 0.1% to about 6%, preferably from about 0.5% to about 5%.

Carrier

The carrier of the shampoo composition is predominantly water, but non-aqueous solvents can also be used to solubilize composition ingredients that are not sufficiently soluble in water, to adjust the viscosity of the composition or to act as humectant. Suitable solvents include polyols, like glycols, glycerol or mixtures thereof. The optional non-aqueous solvents should not adversely affect the ability of the composition to cleanse and treat the hair and scalp or adversely affect aesthetic appeal of the formulation. A non-aqueous solvent can be present in the hair shampoo-conditioning composition of the present invention in an amount ranging from 0% to about 5% by weight of the composition. The shampoos herein are preferably in the form of liquids or creams in which water is the principal diluent. The level of water in the compositions is typically from about 35% to about 90% by weight.

Optional Ingredients

The antidandruff shampoos herein can contain a variety of non-essential optional ingredients for rendering such compositions more stable and desirable. Such conventional optional ingredients are well known to those skilled in the art, e.g., preservatives such as benzyl alcohol, methyl paraben, propyl paraben, and imidazoline urea; thickeners and viscosity modifiers such as coconut ethanol amide, sodium chloride, sodium sulfate, carboxymethylcellulose, methyl cellusose, polyvinyl alcohol, and ethyl alcohol; pH adjusting agents such as citric acid, succinic acid, phosphoric acid, sodium hydroxide, sodium carbonate, etc.; suspending agents such as magnesium/aluminum silicate; perfumes, dyes; and, sequestering agents such as disodium ethylenediamine tetraacetate.

Minor ingredients such as perfumes, dyes and coloring agents can also be added to the instant compositions to improve their consumer acceptability. If present, such agents generally compromise from about 0.1% to 2.0% by weight of the composition. The pH of the shampoo herein is generally from about 3 to about 9.

Preservatives

Preservatives are required in shampoos to safeguard against microbial action that could cause spoilage of the product. Commonly used preservatives in shampoos include methyl and propyl para-hydroxybenzoates alone or in combination with imidazolinyl urea, methylisothioazolinone, methyloldiethylhydantoin (DMDMH), methchloroisothiazolinone, and N-(3-chloroallyl)hexaminium chloride (Quaternium 15).

Other Hair Care Compositions

Crème rinses, hair tonics and other hair care compositions as well as shampoos may contain pyritheon salts and tea tree oil of the present invention. These compositions may contain a variety of other components such as menthol, horsetail extract, nettle extract and others as described above.

pH

The pH of the present compositions is generally not critical and may be obtained through the proper selection of surfactants or through the use of buffer systems such as citric acid/sodium citrate. Improved composition and color stability is, however, achieved by maintaining the pH within the range of from about 5.5 to about 7.5 preferably from about 6.0 to 7.0.

Method of Use

The present antidandruff shampoo compositions are used in a conventional manner for cleaning hair and controlling dandruff on the scalp. The compositions hereof can also be effective for cleaning and controlling malodor associated with the skin. An effective amount of the composition, typically from about 1 gram to 20 gram of the composition, for cleaning hair, scalp, and other region of the body.

Application of the hair typically includes working the composition through the hair and scalp such that most or all of the hair and scalp is contacted with the composition.

The following examples further describe and demonstrate the preferred embodiments within the scope of the present invention. The examples are given solely for the purpose of illustration and are not to be construed as limitations of the present invention as many variations thereof are possible without departing from its spirit and scope.

Standard Preparation of Antidandruff Conditioning Shampoo

The compositions are prepared as follows:

A known amount of DI (deionized) water was added in the beaker followed by the addition of the citric acid and a surfactant with continuous mixing for about 30 minutes at a moderate speed. Next, the beaker is heated to 75 to 80° C. and the additional surfactant compositions are added. Mixing is continued for about 30 minutes. Next, the batch is cooled to about 60° C. and additional surfactants and the natural oils, if used, are added. Mixing is continued for about 30 minutes or until the batch is uniform. In a separate beaker, the color ingredients are dissolved in water, and these are mixed until all color is dissolved. Next, the pre-mixed color is mixed into main batch. Next the glycerin is added to the main batch and it continues to be mixed for about 30 minutes. The batch is then cooled to about 45° C. and additional ingredients are added, while in a separate beaker, the anti-dandruff ingredient is mixed with DI water for about 20 minutes. Next, the anti-dandruff premix is added to the main batch and the DMDM Hydrantoin is mixed with the main batch for about 20 minutes to produce the final composition.

The shampoo composition prepared according to the procedure described above has the following characteristics:

| | |
|---|---|
| Appearance: | Opaque viscous liquid |
| Odor: | Medicinal |
| Color: | Light blue |
| pH @ 25° C.: | 6.2-7.0 |
| Specific gravity: | 0.96-1.04 |
| Viscosity: | 7,000-13,000 cPs |
| @ 25° C., Brookfield Model LVT, | |
| Spindle No. 4 @ 12 RPM | |

### EXAMPLES

In order to study the effect of Tea Tree oil and zinc omadine, three shampoos were prepared using the method stated above to make the compositions shown in Table 1 as Examples 1, 2, and 3 respectively. These examples effectively compare the use of tea tree oil alone versus the use of zinc pyrithione alone versus the use of tea tree oil and zinc pyrithione in combination.

TABLE 1

| | | Ex- | Ex- | Ex- |
|---|---|---|---|---|
| Item # | Ingredient | ample 1 | ample 2 | ample 3 |
| 1 | D I water | 48.45 | 51.65 | 47.45 |
| 2 | Citric acid | 0.10 | 0.10 | 0.10 |
| 3 | Polyquaternium 10 | 0.45 | 0.45 | 0.45 |
| 4 | Cocamidopropyl Betaine | 3.0 | 3.0 | 3.0 |
| 5 | Lauramide DEA | 2.0 | 2.0 | 2.0 |
| 6 | Olealkonium chloride | 1.0 | 1.0 | 1.0 |
| 7 | Glycol distearate | 1.8 | 1.8 | 1.8 |
| 8 | Cocamidopropyl dimethylamine | 3.0 | 3.0 | 3.0 |
| | proprionate | | | |
| 9 | Cetrimonium chloride | 0.5 | 0.5 | 0.5 |
| 10 | Disodiumdimethicone copolyol | 0.80 | 0.80 | 0.80 |
| | sulfosuccinate | | | |
| 11 | Glycol distearate/sodium laureth | 1.0 | 1.0 | 1.0 |
| | sulfate/cocamide MEA/Laureth-10 | | | |
| 12 | Sodium lauryl sulfate | 30 | 30 | 30 |
| 13 | Menthol crystals | 0.2 | 0.2 | 0.2 |
| 14 | Tea Tree Oil | 0.0 | 1.0 | 1.0 |
| 15 | FD & C Blue #1 | 0.0006 | 0.0003 | 0.006 |
| 16 | Glycerin | 1.5 | 1.5 | 1.5 |
| 17 | Hydrolyzed keratin | 1.0 | 1.0 | 1.0 |
| 18 | Horse tail extract | 0.3 | 0.3 | 0.3 |
| 19 | Nettle extract | 0.3 | 0.3 | 0.3 |
| 20 | D I water | 2.1 | | 2.1 |
| 21 | Zinc Omadine (48% active) | 2.1 | | 2.1 |
| 22 | DMDM Hydantoin | 0.4 | 0.4 | 0.4 |

In order to demonstrate the present invention of a combination of antidandruff ingredient plus tea tree oil in a conditioning shampoo (Example 3), as compared to samples containing only the antidandruff ingredient (Example 1) or only the tea tree oil (Example 2), these compositions were tested in a conventional manner for cleaning hair and controlling dandruff on the scalp. Several volunteers having dandruff and scalp itching problems were selected to use the shampoo in a conventional manner over a period of 4-6 weeks. When used, an effective amount of the composition, depending upon the amount and thickness of the hair, typically from 2 g to about 20 g of the composition, was applied to the hair or scalp that has preferably been wetted, generally with water, worked through the hair and scalp and rinsed. The application of compositions was repeated if desired. The compositions were applied at least two to three times a week, preferable once a day. At the end of test period the effectiveness of compositions as antidandruff was checked for dandruff on the scalp and dandruff particles on the hair samples by observation using a video microscope. The results of these tests showed that composition of the present invention performed better than the use of antidandruff ingredient alone and better than tea tree oil alone. These results support the new and unexpected results obtained with composition containing antidandruff agent and tea tree oil, in terms of a cleaner scalp, reduced itching and reduced or elimination of dandruff as compared to compositions using an antidandruff ingredient alone or tea tree oil alone. It is expected, based upon its recognized activity, that the use of tea tree oil will result in a product that has antifungal activity, anti inflammatory effects, as well as anti itch activity, due to reduced dryness.

Thus, it can be seen that the objects of the invention have been satisfied by the structure and its method for use presented above. While in accordance with the Patent Statutes, only the best mode and preferred embodiment has been presented and described in detail, it is to be understood that the invention is not limited thereto or thereby.

What is claimed is:

1. An antidandruff conditioning shampoo comprising a cleansing surfactant, a water-insoluble antidandruff and/or anti-itching agent, a conditioning agent, tea tree oil, and a suitable carrier.

2. The shampoo of claim 1 wherein the tea tree oil is present in an amount of 0.5 % to 10% by weight based upon the weight of the composition.

3. The shampoo of claim 1 wherein the surfactant comprises at least one anionic surfactant.

4. The shampoo of claim 1 wherein the shampoo comprises:
(a) 10% to 45% by weight, based upon the total weight of the shampoo, of a synthetic surfactant;
(b) 0.01% to 5% by weight, based upon the total weight of the shampoo, a water-insoluble antidandruff and/or anti-itching agent
(c) 0.1% to 5% by weight, based upon the total weight of the shampoo, a conditioning agent
(d) 0.01% to 5.0% by weight, based upon the total weight of the shampoo, tea tree oil, and
(e) enough suitable carrier to total 100%.

5. The shampoo of claim 1 wherein the suitable carrier is water.

6. The shampoo of claim 1 wherein the shampoo additionally contains compositions selected from the group consisting of foam boosters, foam stabilizers, preservatives, acid and/or base buffers, non-aqueous solvents, and combinations thereof.

7. The shampoo of claim 1 wherein the conditioning agent comprises a mixture of agents and contains at least one quaternized polymer.

8. The shampoo of claim 1 wherein the antidandruff agent is zinc pyrithione.

9. The shampoo of claim 1 wherein the antidandruff agent is zinc pyrithione and it is present in an amount of about 1.0% and the tea tree oil is present in an amount of about 1.0%.

10. A method of making an antidandruff conditioning shampoo comprising mixing:
(a) a cleansing surfactant,
(b) a water-insoluble antidandruff and/or anti-itching agent,
(c) a conditioning agent,
(d) tea tree oil, and
(e) a suitable carrier.
